(19) United States
(12) Patent Application Publication
(10) Pub. No.: US 2026/0198950 A1
(43) Pub. Date: Jul. 16, 2026

(54) ASPIRATION SHEATH WITH INTEGRATED DILATOR AND ASPIRATION SLEEVE

(71) Applicant: Boston Scientific Scimed, Inc., Maple Grove, MN (US)

(72) Inventors: Joseph Stanley Czyscon, Elk River, MN (US); Derek Kenneth Larson, Golden Valley, MN (US)

(73) Assignee: Boston Scientific Scimed, Inc., Maple Grove, MN (US)

(21) Appl. No.: 19/447,401

(22) Filed: Jan. 13, 2026

Related U.S. Application Data
(60) Provisional application No. 63/744,992, filed on Jan. 14, 2025.

(51) Int. Cl. A61B 17/221; A61M 1/00; A61M 29/02; A61B 17/00; A61B 17/22

(52) U.S. Cl. CPC A61B 17/221; A61M 1/743; A61M 1/815; A61M 1/86; A61M 29/02; A61B 2017/00561; A61B 2017/22079; A61B 2017/2215

(57) ABSTRACT

An aspiration sheath system for aspirating occlusive material from a blood vessel is disclosed. The aspiration sheath system may be configured to have the ability to be unclogged, while retaining the position of the aspiration sheath in the target vasculature. The system includes an aspiration sheath including an elongate shaft extending distally from a manifold, an aspiration sleeve having an elongate shaft slidably disposed within a lumen of the aspiration sheath, and a dilator having an elongate shaft slidably disposed within an aspiration lumen of the aspiration sleeve.

### CROSS-REFERENCE TO RELATED APPLICATION

This application claims the benefit of U.S. Provisional Patent Application Serial No. 63/744,992, filed January 14, 2025, entitled " ASPIRATION SHEATH WITH INTEGRATED DILATOR AND ASPIRATION SLEEVE”, which is incorporated by reference herein in its entirety.

### TECHNICAL FIELD

The present disclosure relates generally to medical devices and more particularly to aspiration sheaths that are adapted for aspirating emboli and/or thrombus.

### BACKGROUND

In some instances, it may be desirable to use an aspiration sheath for aspirating emboli and/or thrombus from a vasculature. However, in the event the aspiration sheath becomes clogged, the aspiration sheath must be removed from the patient to remove the obstruction and/or exchange the aspiration sheath for another one. Therefore, it may be desirable to design an aspiration sheath having the ability to be unclogged, while retaining the position of the aspiration sheath in the target vasculature.

### BRIEF SUMMARY

This disclosure provides design, material, systems, manufacturing method, and use alternatives for dilators, sheaths, sleeves and other related devices and device systems. An example medical device system of the present disclosure includes an aspiration sheath system. The system includes an aspiration sheath, an aspiration sleeve, and a dilator. The aspiration sheath includes an elongate shaft extending distally from a manifold. The aspiration sheath has a lumen extending therethrough. The aspiration sleeve has an elongate shaft slidably disposed within the lumen of the aspiration sheath. The aspiration sleeve has an aspiration lumen extending therethrough. The dilator has an elongate shaft slidably disposed within the aspiration lumen of the aspiration sleeve.

Alternatively or additionally to any of the examples disclosed herein, the aspiration sleeve includes a hub at a proximal end of the elongate shaft of the aspiration sleeve.

Alternatively or additionally to any of the examples disclosed herein, the system includes a syringe fluidly coupled to the hub of the aspiration sleeve for aspiration through the aspiration lumen.

Alternatively or additionally to any of the examples disclosed herein, the system includes a valve positioned between the syringe and the hub.

Alternatively or additionally to any of the examples disclosed herein, the aspiration sleeve includes an expandable funnel at a distal end thereof.

Alternatively or additionally to any of the examples disclosed herein, the expandable funnel is configured to flare outwardly in a distal direction.

Alternatively or additionally to any of the examples disclosed herein, the expandable funnel is positionable distal of a distal end of the elongate shaft of the aspiration sheath to permit the expandable funnel to self-expand.

Alternatively or additionally to any of the examples disclosed herein, the aspiration lumen of the aspiration sleeve has a diameter of 8 French or more.

Alternatively or additionally to any of the examples disclosed herein, the elongate shaft of the aspiration sheath has an outer diameter of 16 French or more.

Another example is an aspiration sheath system. The system includes an aspiration sheath, an aspiration sleeve, and a vacuum source. The aspiration sheath includes an elongate shaft extending from a manifold to a distal end. The elongate shaft has a lumen extending therethrough. The aspiration sleeve has an elongate shaft and a hub at a proximal end thereof. The elongate shaft of the aspiration sleeve is configured to be inserted through the lumen of the aspiration sheath. The vacuum source is fluidly coupled to the hub of the aspiration sleeve for providing aspiration through an aspiration lumen of the aspiration sleeve.

Alternatively or additionally to any of the examples disclosed herein, the system includes a valve positioned between the vacuum source and the hub.

Alternatively or additionally to any of the examples disclosed herein, the vacuum source comprises a syringe.

Alternatively or additionally to any of the examples disclosed herein, the aspiration sleeve includes an expandable funnel at a distal end thereof.

Alternatively or additionally to any of the examples disclosed herein, the expandable funnel is configured to flare outwardly in a distal direction.

Alternatively or additionally to any of the examples disclosed herein, the elongate shaft of the aspiration sheath has an outer diameter of 20 French or greater.

Another example is a method of aspirating occlusive material from a blood vessel. The method includes advancing an aspiration sheath having an aspiration sleeve and a dilator disposed therein to a treatment site within a blood vessel; withdrawing the dilator from the aspiration sleeve while maintaining the aspiration sleeve within the aspiration sheath; creating suction through the aspiration sleeve to aspirate occlusive material from the blood vessel; withdrawing the aspiration sleeve from the aspiration sheath while maintaining the position of the aspiration sheath at the treatment site; and creating suction through the aspiration sheath to continue aspirating occlusive material from the blood vessel.

Alternatively or additionally to any of the examples disclosed herein, advancing the aspiration sheath includes advancing a tapered distal tip of the dilator beyond a distal end of the aspiration sheath.

Alternatively or additionally to any of the examples disclosed herein, creating suction through the aspiration sleeve includes actuating a syringe fluidly coupled to a hub of the aspiration sleeve.

Alternatively or additionally to any of the examples disclosed herein, the method includes advancing a distal end of the aspiration sleeve beyond a distal end of the aspiration sheath prior to creating suction through the aspiration sleeve.

Alternatively or additionally to any of the examples disclosed herein, the aspiration sleeve includes an expandable funnel at a distal end thereof, the method further comprising expanding the expandable funnel distal of the distal end of the aspiration sheath.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of preferred embodiments of the present disclosure. It should be noted that the figures are not drawn to scale and that elements of similar structures or functions are represented by like reference numerals throughout the figures. In order to better appreciate how the above-recited and other advantages and objects of the present disclosure are obtained, a more particular description of the present disclosure briefly described above will be rendered by reference to specific embodiments thereof, which are illustrated in the accompanying drawings. Understanding that these drawings depict only exemplary embodiments of the disclosure and are not therefore to be considered limiting of its scope, the present disclosure will be described and explained with additional specificity and detail through the use of the accompanying drawings, in which:

FIG. 1 illustrates a side view of an aspiration sheath system, including an aspiration sheath, as well as a dilator and aspiration sleeve associated therewith.

FIG. 2 is a cross-sectional view of the aspiration sheath system of FIG. 1 taken along line 2-2.

FIG. 3A illustrates a side view of the dilator and aspiration sleeve of the aspiration sheath system of FIG. 1.

FIG. 3B illustrates a side view of an aspiration sleeve of the aspiration sheath system of FIG. 1.

FIG. 3C illustrates a side view of an alternative aspiration sleeve of the aspiration sheath system of FIG. 1.

FIGS. 4-6 illustrate an exemplary method of using the aspiration sheath system of FIG. 1.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this disclosure and appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following detailed description should be read with reference to the drawings in which similar elements in different drawings are numbered the same. The drawings, which are not necessarily to scale, depict illustrative examples and are not intended to limit the scope of the disclosure.

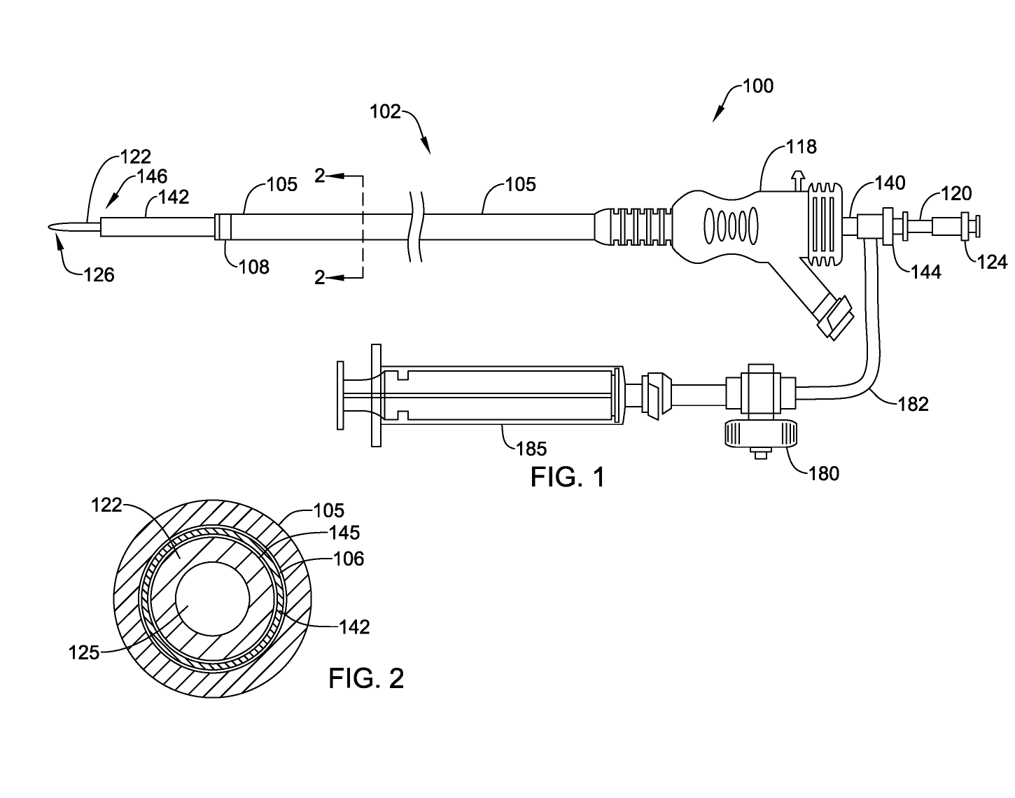

As discussed above, it may be desirable to design an aspiration sheath having the ability to be unclogged, while retaining the position of the aspiration sheath in the target vasculature. FIG. 1 is a side view of components of an example aspiration sheath system 100 of the present disclosure. As shown in FIG. 1, the aspiration sheath system 100 may include an aspiration sheath 102 having an elongate shaft 105 extending distally from a manifold 118. The elongate shaft 105 may have a proximal end extending to and coupled or otherwise connected to the manifold 118. The elongate shaft 105 may extend distally from the manifold 118 to a distal end 108 of the elongate shaft 105. A lumen (not shown in FIG. 1) may extend through the elongate shaft 105 from the proximal end to the distal end, and thus the lumen may extend along an entire length of the elongate shaft 105 from the proximal end to the distal end of the elongate shaft 105. The elongate shaft 105 may include a single lumen or a plurality of lumens disposed concentrically, coaxially, in a side-by-side relationship, in an array, or in any configuration feasible for aspiration of emboli and/or thrombus, and/or for the passage of medical devices and/or like devices therethrough. The distal end of the elongate shaft 105 may further include a distal tip defining a distal opening into the lumen of the elongate shaft 105. In some instances, the distal tip may include radiopaque material. For instance, and applicable to all examples of the present disclosure, radiopaque material may be applied to the distal tip or any other part of the elements disclosed herein through any desired process.

As shown in FIG. 1, the elongate shaft 105 of the aspiration sheath 102 may have any desired outer diameter. For instance, the elongate shaft 105 may have a relatively large outer diameter or French (Fr) size, such as 20 Fr. or greater, 22 Fr. or greater, or 24 Fr. or greater. However, other French sizes are contemplated for the outer diameter of the elongate shaft 105 of the aspiration sheath 102. The lumen through the elongate shaft 105 may have a constant diameter sized to receive other components of the aspiration sheath system, as described herein.

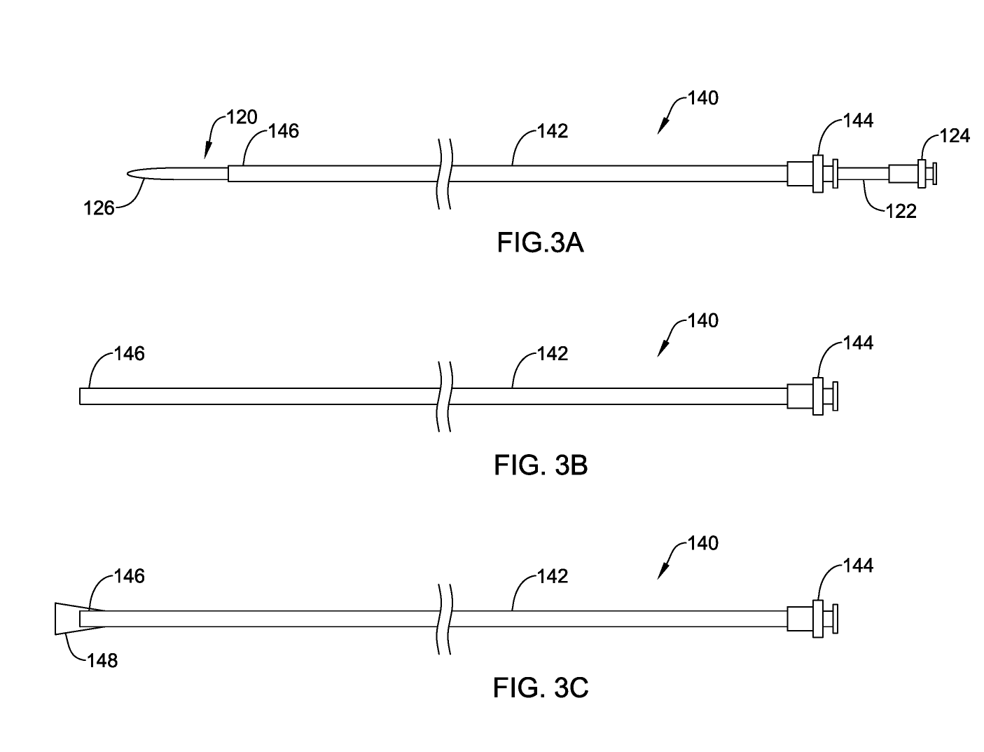

The aspiration sheath system 100 may also include a dilator 120 and an aspiration sleeve 140, also shown in FIG. 1, configured to be inserted through the lumen of the elongate shaft 105 of the aspiration sheath 102. The combination of the dilator 120 and the aspiration sleeve 140 is also shown in FIG. 3A which illustrates a side view of the dilator 120 and aspiration sleeve 140 of the aspiration sheath system of FIG. 1. The dilator 120 may facilitate introduction of the aspiration sheath 102 into the vasculature and subsequent advancement of the aspiration sheath 102 through the vasculature of the patient. The dilator 120 may include an elongate shaft 122 extending distally from a hub 124 to a distal tip 126 of the dilator 120. The elongate shaft 122 of the dilator 120 may extend distally beyond the distal end of the elongate shaft 105 while the hub 124 is positioned proximal of the manifold 118. For example the dilator 120 may include a tapered distal tip 126 extendable distally beyond the distal tip of the elongate shaft 105 of the aspiration sheath 102 to facilitate delivery of the aspiration sheath 102 through the vasculature of a patient.

The aspiration sleeve 140 of the aspiration sheath system 100 is also configured to be inserted through the lumen of the elongate shaft 105 of the aspiration sheath 102 with the dilator 120 extending through the lumen of the aspiration sleeve 140. The aspiration sleeve 140 is further illustrated in FIG. 3B which illustrates a side view of the aspiration sleeve 140 of the aspiration sheath system of FIG. 1. The aspiration sleeve 140 may include an elongate shaft 142 extending or extendable distally from a hub 144 to a distal end 146 of the aspiration sleeve 140. In some instances, the elongate shaft 142 of the aspiration sleeve 140 may extend distally beyond the distal tip of the elongate shaft 105 of the aspiration sheath 102 while the hub 144 is positioned proximal of the manifold 118. In other instances, the distal end 146 of the elongate shaft 142 of the aspiration sleeve 140 may be flush with or proximal of the distal tip of the elongate shaft 105 of the aspiration sheath 102 when disposed therein.

FIG. 2 is a cross-sectional view of the elongate shaft 105 of the aspiration sheath 102 taken along line 2-2 of FIG. 1 with the elongate shaft 142 of the aspiration sleeve 140 and the elongate shaft 122 of the dilator 120 inserted therein. The elongate shaft 142 of the aspiration sleeve 140 may be positioned within a lumen 106 of the elongate shaft 105 of the aspiration sheath 102, and the elongate shaft 122 of the dilator 120 may be positioned within a lumen 145 of the elongate shaft 142 of the aspiration sleeve 140. The dilator 120 may include a guidewire lumen 125 extending through the elongate shaft 122 of the dilator for accommodating a guidewire therethrough. The guidewire lumen 125 may have a diameter in the range of 0.38 millimeters (mm) (0.015 inches) to 1.02 mm (0.040 inches), to accommodate 0.36 mm (0.014 inch) to 0.97 mm (0.038 inch) diameter guide wires, for example. Accordingly, the elongate shaft 142 of the aspiration sleeve 140 may surround the elongate shaft 122 of the dilator 120, with the elongate shaft 122 of the dilator 120 longitudinally moveable within the lumen 145 of the aspiration sleeve 140. Furthermore, the elongate shaft 105 of the aspiration sheath 102 may surround the elongate shaft 142 of the aspiration sleeve 140, with the elongate shaft 142 of the aspiration sleeve 140 longitudinally moveable within the lumen 106 of the aspiration sheath 102.

As will be described further herein, after inserting the aspiration sheath 102, with the dilator 120 and aspiration sleeve 140 inserted therethrough, into a vasculature to a desired treatment location, the dilator 120 may be removed from the lumen of the elongate shaft 142 of the aspiration sleeve 140 to provide an aspiration lumen (defined by the lumen of the aspiration sleeve 140) through the aspiration sheath 102. The aspiration lumen 145 of the aspiration sleeve 140 may have a diameter of 8 Fr. (2.67 mm) or more, 10 Fr. (3.33 mm) or more, 12 Fr. (4 mm) or more, 15 Fr. (5 mm) or more, 18 Fr. (6 mm) or more, or 20 Fr. (6.67 mm) or more, for example. The aspiration lumen 145 of the aspiration sleeve 140 may have a diameter in the range of 8 Fr. (2.67 mm) to 24 Fr. (8 mm), for example.

The aspiration sheath system 100 may also include a syringe 185, connected to a port of the hub 144 of the aspiration sleeve 140 via a length of tubing 182. A valve 180, such as a stop cock valve, may be positioned between the syringe 185 and the tubing 182 that may be selectively opened and closed to fluidly couple the syringe 185 to the lumen of the tubing 182, and thus fluidly couple the syringe 185 to the lumen 145 of the elongate shaft 142 of the aspiration sleeve 140. The syringe 185 may be used to create a vacuum for aspirating emboli and/or thrombi through the aspiration sleeve 140 and into the syringe 185.

An alternative example of the aspiration sleeve 140 is shown in FIG. 3C which illustrates a side view of an alternative aspiration sleeve of the aspiration sheath system of FIG. 1. In some instances, the aspiration sleeve 140 may include an expandable funnel 148 at the distal end 146 of the elongate shaft 142. In some instances, the expandable funnel 148 may be a braided or woven construct formed of a plurality of interwoven filaments. In some instances, the expandable funnel 148 may be an extruded member or a laser cut construct (e.g., a monolithic construct) formed of a self-expandable material, such as nitinol.

The expandable funnel 148 may flare outwardly in a distal direction from the elongate shaft 142 to a diameter greater than the outer diameter of the elongate shaft 105 of the aspiration sheath 102 to provide an enlarged distal opening into the lumen 145 of the elongate shaft 142. The expandable funnel 148 may facilitate aspirating emboli or thrombus into the lumen 145 of the aspiration sleeve 140. During delivery of the aspiration sheath 102 to a target location, the expandable funnel 148 may be constrained in a collapsed configuration within the lumen 106 of the elongate shaft 105 of the aspiration sheath 102. When desired, the expandable funnel 148 may be deployed from the distal end of the elongate shaft 105 of the aspiration sheath 102 to allow the expandable funnel 148 to radially expand to its radially expanded configuration. In some instances, the expandable funnel 148 may be advanced distally of the distal end of the elongate shaft 105 of the aspiration sheath 102 by a distance of 2 cm (0.79 inches) or more, or by a distance of 2 cm or more, when deployed. In some instances, the expandable funnel 148 may be advanced distally of the distal end of the elongate shaft 105 of the aspiration sheath 102 by a distance of 4 cm (1.57 inches) or less, by a distance of 3 cm (1.18 inches) or less, or by a distance of 2 cm or less, when deployed. In some instances, the expandable funnel 148 may be advanced distally of the distal end of the elongate shaft 105 of the aspiration sheath 102 by a distance of 2 cm to 4 cm, or by a distance of 2 cm to 3 cm, when deployed. The expandable funnel 148 may allow for increased aspiration capability of the aspiration sleeve 140.

In some instances, the expandable funnel 148 may be configured to automatically self-expand when advanced distal of the distal end of the elongate shaft 105 of the aspiration sheath 102, and thus unconstrained by the aspiration sheath 102. In other instances, the expandable funnel 148 may be actuatable by the user, such as with a pull wire, etc., to radially expand the expandable funnel 148 distal of the distal end of the elongate shaft 105 of the aspiration sheath 102 at a target location.

Once the expandable funnel 148 has captured the target occlusive material (e.g., emboli or thrombi) and/or aspiration is completed, the expandable funnel 148 may be retracted back into the elongate shaft 105 of the aspiration sheath 102 prior to withdrawing the aspiration sheath 102 and the aspiration sleeve 140 from the patient.

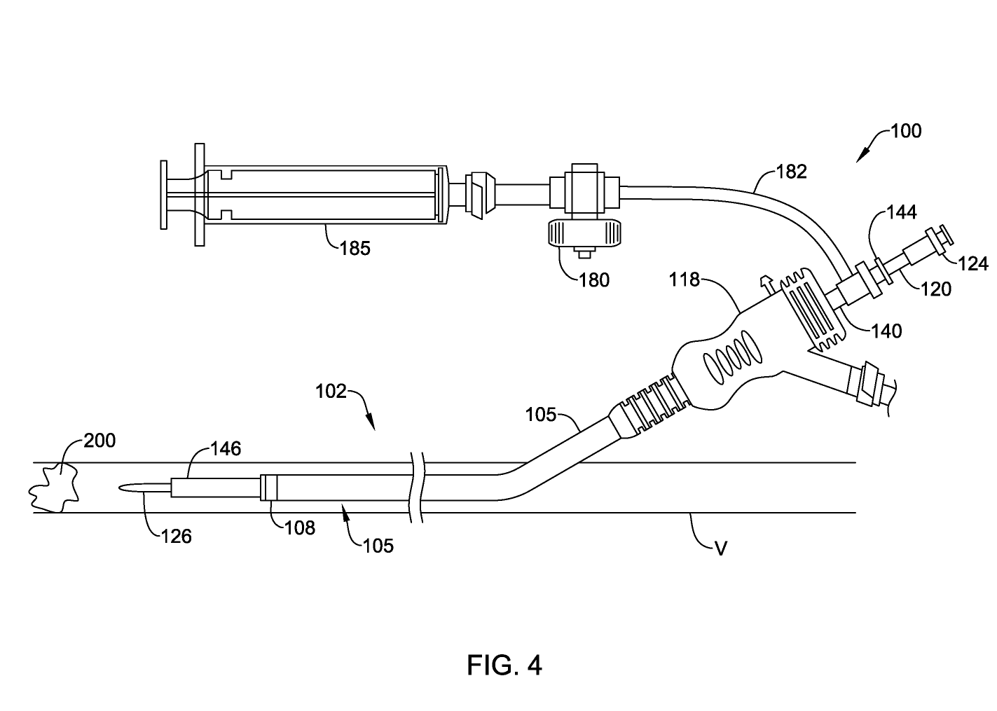

FIGS. 4-6 illustrate a sequence of steps of utilizing the aspiration sheath system 100. FIG. 4 is a side view of the aspiration sheath system 100 of the present disclosure as the elongate shaft 105 of the aspiration sheath 102 is positioned within a blood vessel V of a patient or subject with the dilator 120 and aspiration sleeve 140 disposed therein and a distal tip 126 of the dilator 120 extending distally beyond the distal end of the elongate shaft 105 of the aspiration sheath 102, and distally beyond the distal end 146 of the elongate shaft 142 of the aspiration sleeve 140. The aspiration sheath system 100 may be deployed within the blood vessel V (e.g., by tracking along a guidewire already positioned within the blood vessel V), and may be advanced into the blood vessel V. The aspiration sheath system 100 may be advanced within the blood vessel V until the distal end of the aspiration sheath system 100 is proximate an occlusion 200 (e.g., emboli, thrombus, etc.).

Once advanced to a desired location within the blood vessel V, the dilator 120 may be retracted and removed from the elongate shaft 105 of the aspiration sheath 102 (i.e., removed from the lumen 145 of the elongate shaft 142 of the aspiration sleeve 140, leaving the aspiration sleeve 140 disposed within the lumen 106 of the elongate shaft 105 of the aspiration sheath 102. In some instances, the elongate shaft 142 of the aspiration sleeve 140 may extend to the distal end of the elongate shaft 105 or distally beyond the distal end of the elongate shaft 105 while the hub 144 is positioned proximal of the manifold 118. In some instances, once navigated to a treatment site within the blood vessel V, the aspiration sleeve 140 may be advanced distally relative to the elongate shaft 105 of the aspiration sheath 102 to advance the distal end region of the elongate shaft 142 of the aspiration sleeve 140 distally beyond the distal end 108 of the elongate shaft 105 of the aspiration sheath 102. In instances in which the aspiration sleeve 140 includes an expandable funnel at the distal end thereof, the expandable funnel may be expanded within the blood vessel V distal of the distal end 108 of the elongate shaft 105 of the aspiration sheath 102.

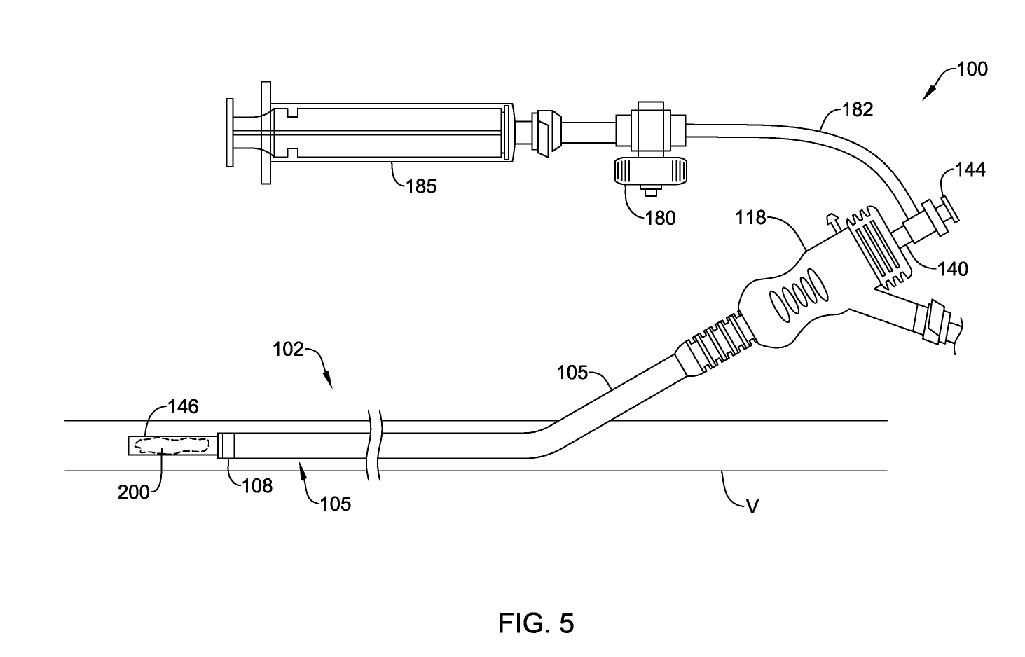

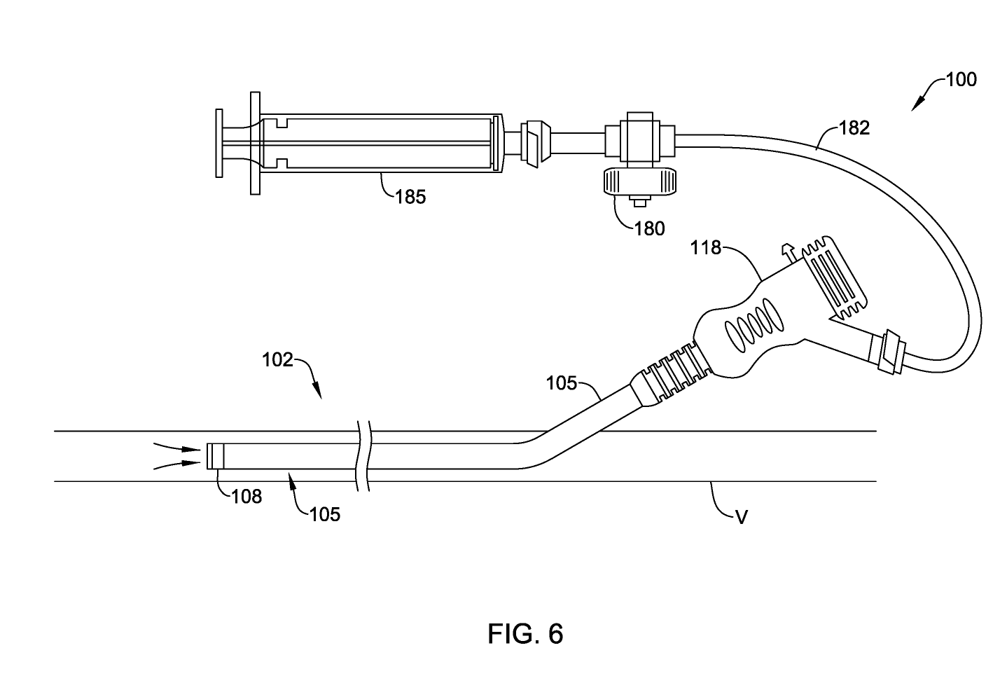

Thereafter, the syringe 185, or another syringe or other vacuum device, which may be connected to the hub 144 of the aspiration sleeve 140, may be used to aspirate the occlusion 200 (e.g., emboli and/or thrombus) through the lumen 145 of the aspiration sleeve 140 and thus the aspiration sheath 102 into the syringe 185. As shown in FIG. 5, which is a side view of the aspiration sheath system 100 of the present disclosure, if an occlusion 200 becomes clogged within the lumen 145 of the aspiration sleeve 140 during the medical procedure, the aspiration sleeve 140 may be proximally withdrawn from the aspiration sheath 102 while the aspiration sheath 102 remains positioned in the blood vessel V at the treatment site. Accordingly, as shown in FIG. 6, which is a side view of the aspiration sheath system 100 of the present disclosure, aspiration of the treatment site may be continued with the aspiration sheath 102 without the aspiration sleeve 140. Namely, the syringe 185, or another syringe or other vacuum device, may be connected to a port of the manifold 118 of the aspiration sheath 102 (which is in fluid communication with the lumen 106 of the elongate shaft 105 of the aspiration sheath 102) and be used to aspirate further occlusive material directly through the lumen 106 of the elongate shaft 105 of the aspiration sheath 102 into the syringe 185.

In at least some examples, portions or all of the various components of the aspiration sheath system 100 may include a reinforcement structure, such as a braided reinforcement layer, a coiled reinforcement layer, or the like.

In at least some examples, portions or all of the various components of the aspiration sheath system 100 may also be doped with, made of, or otherwise include a radiopaque material including those listed herein or other suitable radiopaque materials. In some embodiments, a degree of MRI compatibility is imparted into the aspiration sheath system 100. For example, to enhance compatibility with Magnetic Resonance Imaging (MRI) machines, it may be desirable to make the various components of the aspiration sheath system 100 in a manner that would impart a degree of MRI compatibility. For example, the various components of the aspiration sheath system 100 may be made of a material that does not substantially distort the image and create substantial artifacts (artifacts are gaps in the image). Certain ferromagnetic materials, for example, may not be suitable because they may create artifacts in an MRI image. The various components of the aspiration sheath system 100 may also be made from a material that the MRI machine can image. Some materials that exhibit these characteristics include, for example, tungsten, cobalt-chromium-molybdenum alloys, nickel-cobalt-chromium-molybdenum alloys, nitinol, and the like, and others.

In some embodiments, the exterior surface of the aspiration sheath system 100 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve ease of advancement within the vasculature. Suitable lubricious polymers may include silicone and the like, polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility.

Some examples of suitable polymers and/or elastomers and/or elastomeric material that may be used with any of the examples disclosed herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be noted and can be appreciated that some of the figures are schematic in nature and are not drawn to scale. Certain features are shown larger than their scale and certain features are omitted from some views for ease of illustration.

It should also be noted that, as used in this specification and the appended claims, the singular forms include the plural unless the context clearly dictates otherwise. It should also be noted that the term “or” is generally employed in its sense including “and/or” unless the content clearly dictates otherwise.

The present disclosure has been described with reference to various specific and exemplary embodiments. Those skilled in the art will understand that changes may be made in details, particularly in matters of shape, size, material and arrangement of parts. Accordingly, various modifications and changes may be made to the examples and the embodiments. Additional or fewer components may be used, depending on the condition that is being treated by the electrosurgical ablation device and other related devices and components disclosed herein. It should be understood that many variations and modifications may be made while remaining within the spirit and scope of the present disclosure. The specifications and drawings are, therefore, to be regarded in an illustrative rather than a restrictive sense.

What is claimed is:

1. An aspiration sheath system comprising:
an aspiration sheath including an elongate shaft extending distally from a manifold, the aspiration sheath having a lumen extending therethrough;
an aspiration sleeve having an elongate shaft slidably disposed within the lumen of the aspiration sheath, the aspiration sleeve having an aspiration lumen extending therethrough; and
a dilator having an elongate shaft slidably disposed within the aspiration lumen of the aspiration sleeve.

2. The aspiration sheath system of claim 1, wherein the aspiration sleeve includes a hub at a proximal end of the elongate shaft of the aspiration sleeve.

3. The aspiration sheath system of claim 2, further comprising a syringe fluidly coupled to the hub of the aspiration sleeve for aspiration through the aspiration lumen.

4. The aspiration sheath system of claim 3, further comprising a valve positioned between the syringe and the hub.

5. The aspiration sheath system of claim 1, wherein the aspiration sleeve includes an expandable funnel at a distal end thereof.

6. The aspiration sheath system of claim 5, wherein the expandable funnel is configured to flare outwardly in a distal direction.

7. The aspiration sheath system of claim 5, wherein the expandable funnel is positionable distal of a distal end of the elongate shaft of the aspiration sheath to permit the expandable funnel to self-expand.

8. The aspiration sheath system of claim 1, wherein the aspiration lumen of the aspiration sleeve has a diameter of 8 French or more.

9. The aspiration sheath system of claim 8, wherein the elongate shaft of the aspiration sheath has an outer diameter of 16 French or more.

10. An aspiration sheath system comprising:
an aspiration sheath including an elongate shaft extending from a manifold to a distal end, the elongate shaft having a lumen extending therethrough;
an aspiration sleeve having an elongate shaft and a hub at a proximal end thereof, the elongate shaft of the aspiration sleeve configured to be inserted through the lumen of the aspiration sheath; and
a vacuum source fluidly coupled to the hub of the aspiration sleeve for providing aspiration through an aspiration lumen of the aspiration sleeve.

11. The aspiration sheath system of claim 10, further comprising a valve positioned between the vacuum source and the hub.

12. The aspiration sheath system of claim 10, wherein the vacuum source comprises a syringe.

13. The aspiration sheath system of claim 10, wherein the aspiration sleeve includes an expandable funnel at a distal end thereof.

14. The aspiration sheath system of claim 13, wherein the expandable funnel is configured to flare outwardly in a distal direction.

15. The aspiration sheath system of claim 10, wherein the elongate shaft of the aspiration sheath has an outer diameter of 20 French or greater.

16. A method of aspirating occlusive material from a blood vessel, the method comprising:
advancing an aspiration sheath having an aspiration sleeve and a dilator disposed therein to a treatment site within a blood vessel;
withdrawing the dilator from the aspiration sleeve while maintaining the aspiration sleeve within the aspiration sheath;
creating suction through the aspiration sleeve to aspirate occlusive material from the blood vessel;
withdrawing the aspiration sleeve from the aspiration sheath while maintaining the position of the aspiration sheath at the treatment site; and
creating suction through the aspiration sheath to continue aspirating occlusive material from the blood vessel.

17. The method of claim 16, wherein advancing the aspiration sheath includes advancing a tapered distal tip of the dilator beyond a distal end of the aspiration sheath.

18. The method of claim 16, wherein creating suction through the aspiration sleeve includes actuating a syringe fluidly coupled to a hub of the aspiration sleeve.

19. The method of claim 16, further comprising advancing a distal end of the aspiration sleeve beyond a distal end of the aspiration sheath prior to creating suction through the aspiration sleeve.

20. The method of claim 19, wherein the aspiration sleeve includes an expandable funnel at a distal end thereof, the method further comprising expanding the expandable funnel distal of the distal end of the aspiration sheath.
